(19) United States
(12) Patent Application Publication
(10) Pub. No.: US 2026/0349101 A1
(43) Pub. Date: Nov. 5, 2026

(54) Digital 3D Multi-Gate Continuous Wave Mode Using Imaging System on a Chip

(71) Applicant: Exo Imaging, Inc., Santa Clara, CA (US)

(72) Inventors: Kutay Üstüner, Santa Clara, CA (US); Chad Steward, Santa Clara, CA (US); Krishnakumar Sundaresan, Santa Clara, CA (US); Larry Skrenes, Santa Clara, CA (US)

(73) Assignee: Exo Imaging, Inc., Santa Clara, CA (US)

(21) Appl. No.: 19/547,957

(22) Filed: Feb. 24, 2026

Related U.S. Application Data
(60) Provisional application No. 63/767,482, filed on Mar. 5, 2025.

(51) Int. Cl. G01S 7/52; G01S 15/89

(52) U.S. Cl. CPC G01S 7/52031; G01S 7/5209; G01S 15/8979; G01S 15/8993

(57) ABSTRACT

An imaging device can include a transducer that includes an array of piezoelectric elements formed on a substrate. Each piezoelectric element can include at least one membrane suspended from the substrate, at least one bottom electrode disposed on the membrane, at least one piezoelectric layer disposed on the bottom electrode, and at least one top electrode disposed on the at least one piezoelectric layer. Adjacent piezoelectric elements can be configured to be isolated acoustically from each other. The device is utilized to measure flow or flow along with imaging anatomy.

### CROSS REFERENCE TO RELATED APPLICATIONS

This application claims priority to U.S. Provisional Patent Application No. 63/767,482, filed Mar. 5, 2025, the entirety of which is incorporated herein by reference. Also, this application is related to U.S. patent application Ser. No. 17/153,631, filed Jan. 20, 2021 (now U.S. Pat. No. 11,199,623), the entirety of which is also incorporated herein by reference.

### BACKGROUND

### Field of the Inventions

The present inventions relate to a method and system for digital 3D multi-gate Continuous Wave (CW) Mode using an Imaging System on a Chip (iSoC) that is integrated directly on a matrix array of ultrasound transducers.

### Description of the Related Art

Ultrasound imaging utilizes an ultrasound transducer and an imaging system, where the ultrasound transducer comprises an array of pulse-echo sensors or elements. The imaging system consists of a transmit pulser with pulse shaping capability, a transmit beamformer, receive preamplifiers, and a receive beamformer. The output from the receive beamformer may undergo additional processing by downstream processors to extract anatomy and flow information. This processed output can then be displayed on a monitor.

Ultrasound imaging is capable of capturing blood flow information including peak velocity and the relative strengths of velocity components over time within a heart cycle and across heart cycles. Such information proves valuable in assessing cardiovascular hemodynamics, particularly in cases of valvular heart diseases. Two modes employed for obtaining this type of information are Pulsed Wave (PW) Spectral Doppler and Continuous Wave (CW) Spectral Doppler, commonly referred to as PW Doppler and CW Doppler.

### SUMMARY

The PW Doppler Mode uses a finite duration pulse repeatedly transmitted at a user-selectable Pulse Repetition Frequency (PRF) to gather data from the echo of this pulse at a spatial sampling region of interest known as a PW gate. Users can define the line of sight, depth, and axial length of a PW gate through a user interface. This mode's ability to use a finite duration pulse allows it to distinguish echoes from the depth of the PW gate and suppress echoes from depths outside the PW gate. However, PW Doppler mode faces aliasing issues at high flow velocities due to discrete time sampling caused by a finite PRF.

To avoid aliasing CW Doppler Mode is used. A regurgitant jet, indicative of a high-velocity reverse flow through a heart valve defect, can reach considerable speeds, with peak jet velocity and regurgitant volume during systole providing a measure of the defect's severity. However, CW Doppler requires a higher dynamic range receiver frontend than PW modes due to the potential superposition of strong echoes from highly reflective shallow reflectors on the weaker echo of interest from the deeper CW gate. Additionally, using CW Doppler may necessitate reducing transmit acoustic output power to prevent overheating of the transducer surface and surrounding tissue. Furthermore, active transmit and receive apertures may be limited to half the physical aperture to enable simultaneous transmission and reception.

Therefore, at least some disclosed embodiments recognize that addressing the requirements for higher dynamic range and compensation for Signal-to-Noise Ratio (SNR) loss associated with CW Doppler can be achieved by implementing a dedicated analog receive beamformer optimized for CW, running in parallel with a digital receive beamformer optimized for PW modes such as B-Mode, Color Doppler, PW Doppler, and M-Mode. It is acknowledged, according to at least some disclosed embodiments, that this approach could prove effective for traditional ultrasound systems with limited number of transducer elements and acquisition channels. In these cases, analog front-end chips, such as Texas Instruments AFE5828 that use separate and parallel paths dedicated to PW and CW may be used. However, in certain embodiments, such as a digital 3D imaging system on a chip where there are thousands of element and acquisition channels there might be a preference for incorporating CW Doppler capability into the digital path.

As described in further detail below, methods of forming a digital continuous wave ultrasound beam are herein disclosed. In some embodiments, the method comprises, transmitting a CW ultrasound beam from one or more first transducers of an ultrasound imaging system, receiving reflection signals of the CW ultrasound beam at one or more second transducers of the same imaging system to form receive signals, digitizing the receive signals to form digitized receive signals, applying an independent phase rotation to each of the digitized receive signals to form phase rotated digital signals, and summing the phase rotated digital signals to form a digital CW receive beam.

In some embodiments, the one or more first transducers are programmed to perform only a transmit function. Optionally, the one or more second transducers are programmed to perform only a receive function. At least one of the first transducers can be different from at least one of the second transducers. The method can also include the amplifying of the reflection signals before digitizing the reflection signals.

In some embodiments, the method further comprises demodulating the CW receive beam down to baseband signals. In some embodiments, the method further comprises accumulating or low pass filtering and decimating the baseband signals to form input samples of a spectral analyzer.

In some embodiments, the one or more first transducers and the one or more second transducers form a matrix array of transducers. In some embodiments, the one or more first transducers and the one or more second transducers are included in a matrix array of transducers. In some embodiments, the one or more first transducers is a first half of the matrix array of transducers. In some embodiments, the one or more second transducers is a second half of the matrix array of transducers.

In some embodiments, the method further comprises, before transmitting the CW ultrasound beam, applying independent phase rotation to CW excitation of each of the one or more first transducers to steer the CW ultrasound beam substantially along a line of sight of interest and to focus at a depth of interest.

In some embodiments, the applying the independent phase rotation to each of the digitized receive signals is performed to steer the digitized receive signals substantially along a transmit line of sight and focus the digitized receive signals substantially at a transmit focus depth.

In some embodiments, the applying the independent phase rotation to each of the digitized receive signals is performed multiple times in parallel to steer the digitized receive signals along multiple distinct angles and/or depths forming multiple CW receive beams in parallel. In some embodiments, at least one of the multiple CW receive beams is steered out of a standard azimuthal plane at a nonzero angle relative to an axis normal to a probe surface.

In some embodiments, the computing circuit is an application specific integrated circuit (ASIC). In some embodiments, the application specific integrated circuit (ASIC) is integrated with the first one or more transducers and the second one or more transducers.

As disclosed in more detail below, an ultrasound imaging device is disclosed. The device comprises a computing circuit, one or more processors, memory, and one or more programs stored in the memory and configured for execution by the one or more processors, the one or more programs comprising instructions for: transmitting a CW ultrasound beam from one or more first transducers, receiving reflection signals of the CW ultrasound beam at one or more second transducers to form receive signals, digitizing the receive signals to form digitized receive signals, applying an independent phase rotation to each of the digitized receive signals to form phase rotated digital signals, and summing the phase rotated digital signals to form a digital CW receive beam.

In all embodiments one or more first transducers are different from the one or more second transducers.

In some embodiments, the operations further comprise amplifying the reflection signals before digitizing. In some embodiments, the operations further comprise demodulating the CW receive beam down to baseband signals. In some embodiments, the operations further comprise low pass filtering and decimating the baseband signals to form input samples of a spectral analyzer. In some embodiments, the one or more first transducers and the one or more second transducers form a matrix array of transducers. In some embodiments, the one or more first transducers and the one or more second transducers are included in a matrix array of transducers. In some embodiments, the one or more first transducers is a first half of the matrix array of transducers. In some embodiments, the one or more second transducers is a second half of the matrix array of transducers.

In some embodiments, the operations further comprise, before transmitting the CW ultrasound beam, applying independent phase rotation to CW excitation of each of the one or more first transducers to steer the CW ultrasound beam substantially along a line of sight of interest and to focus at a depth of interest.

In some embodiments, the applying the independent phase rotation to each of the digitized receive signals is performed to steer the digitized receive signals substantially along a transmit line of sight and focus the digitized receive signals substantially at a transmit focus depth.

In some embodiments, the applying the independent phase rotation to each of the digitized receive signals is performed multiple times in parallel to steer the digitized receive signals along multiple distinct angles and/or depths forming multiple CW receive beams in parallel. In some embodiments, at least one of the multiple CW receive beams is steered out of a standard azimuthal plane at a nonzero angle relative to an axis normal to a probe surface.

In some embodiments, the computing circuit is an application specific integrated circuit (ASIC). In some embodiments, the application specific integrated circuit (ASIC) is integrated with the first one or more transducers and the second one or more transducers.

### BRIEF DESCRIPTION OF THE DRAWINGS

Various features of illustrative embodiments of the inventions are described below with reference to the drawings. The illustrated embodiments are intended to illustrate, but not to limit, the inventions. The drawings contain the following figures:

FIG. 1 is a block diagram of an imaging system on a chip with digital 3D Multi-Gate CW capability, in accordance with some embodiments of the present disclosure.

FIG. 2 is a flow diagram of a method for continuous wave ultrasound imaging, in accordance with some embodiments of the present disclosure.

### DETAILED DESCRIPTION

The present invention relates to imaging devices, and more particularly to portable handheld ultrasonic imaging devices having the ability to perform flow and anatomy imaging.

In accordance with at least some embodiments disclosed herein is the realization that with the advancements in semiconductor processes it became feasible to integrate a matrix array transducer with a full-array 3D digital transmit and receive beamformer packed in an application specific integrated circuit (ASIC) that has a digital acquisition channel per transducer element, e.g., to create a Full-Array Digital 3D Ultrasound Imaging System Integrated with a Matrix array Transducer. This helps reduce the cost, size, weight, and power of an ultrasound imaging system and improve performance. Such a matrix array may have thousands of transducer elements.

Described herein is a low-cost method and system to add CW Doppler capability to such a digital 3D Imaging System on a Chip (iSoC) without an additional analog receive beamformer path specific to CW Doppler. In accordance with at least some embodiments disclosed herein is the realization that having numerous, e.g., thousands of, digital elements helps compensate at least partially for the loss in Dynamic Range (DNR) and Signal to Noise Ratio (SNR) of CW operation. The chip has 3D focusing capability, which allows the CW gate to be placed anywhere in 3D space. The chip has parallel beamformation (multibeam) capability, which provides the ability to have multiple CW gates simultaneously.

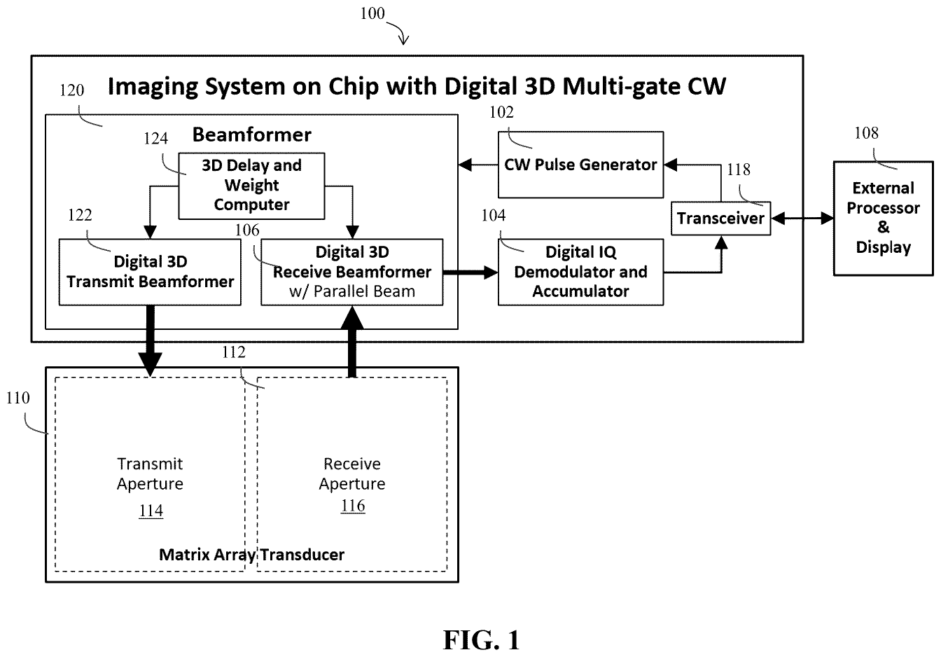

Turning now to FIG. 1, a block diagram of an Imaging System on a Chip (iSoC) with digital 3D Multi-Gate CW capability, in accordance with an embodiment of the present disclosure, is shown.

iSoC 100 may contain an on-chip continuous wave generator 110, a beamformer 120, an analog transmit front-end 130, an analog receive front-end 140, a complex demodulator, accumulator, and decimator 150, and a transceiver 160. The beamformer 120 may contain a 3D Delay and Weight Computer 121, a digital 3D transmit beamformer 122 and a parallel beam capable digital 3D receive beamformer 123. The digital 3D transmit beamformer shifts the phase of the continuous wave signal generated by the continuous wave generator 110 based on the element dependent delay computed by the 3D delay and weight computer 121, weights them by the element dependent weight computed by the same computer. The analog transmit front-end 130 converts the delayed and weighted digital continuous wave signal to an analog continuous wave signal and drives the elements of a transmit aperture 171 of a matrix array 170. The analog receive front-end 140 receives echo signals from the elements of a receive aperture 172 of a matrix array transducer 170, amplifies and filters them. The digital 3D receive beamformer 123 converts the amplified and filtered received echo into a digital signal, shifts the phase of the digital signal based on the element dependent delay computed by the 3D delay and weight computer 121, sums the phase-aligned receive element signals after weighting them individually by the element dependent weight computed by the same computer, forming a CW receive beam. A complex modulator, accumulator, and decimator 150 shifts the CW receive beam to baseband, low pass filters the complex baseband receive beam to filter out signals above the maximum Doppler shift frequency and to improve SNR and decimates to reduce to sampling rate consistent with the bandwidth of the Doppler shift. Transceiver 160 receives control signals and input parameters from an external processor 180 and sends the filtered and decimated baseband CW beams to the external processor 180 for spectral analysis and display. In some embodiments spectral analysis can optionally be performed by an on-chip processor.

iSoC 100 may also be coupled to, e.g., integrated with, matrix array transducer 170. Matrix array transducer 170 may contain a plurality of transducer elements. Each transducer element of matrix array transducer may be connected to a transmit and receive channel of the imaging system. Matrix array transducer may have one or more apertures. In some embodiments, element by element programmable transmit and receive masks may be used to assign half an aperture 171 exclusively to transmit and the other half 172 exclusively to receive for CW Mode. The programmable transmit and receive masks effectively turn off the receive and transmit channels of the transmit aperture 171 and receive aperture 172, respectively, as shown in FIG. 1. The receive and/or transmit channels of a given aperture, being turned off, reduces power consumption and digital interference. The masks can optionally be designed to leave space between the active transmit and receive apertures, such as several columns of elements to reduce acoustic and electronic coupling between transmit and receive.

Continuous wave generator 110 may be triggered by an external processor such as external processor 180, e.g., upon user's request to start CW mode. In some embodiments external processor 180 may communicate with continuous wave generator 110 via a transceiver 160. A center frequency of the continuous wave is preferably programmable. The phase of the continuous wave signal of each transmit element is then rotated independently by digital transmit beamformer 122 to steer and focus the transmit beam to the CW gate position set by the user. The phase rotation of the continuous wave can be accomplished through an element-dependent group delay at the beginning of the continuous wave.

In some embodiments, the transducer array may be a 2D array, and the delay and weight computer, and the transmit and receive beamformers may comprise a 2D beamformer.

An echo received by each receive element of matrix array transducer 172 can first be amplified and band-pass filtered by a low-noise analog receive front-end 140. The amplified and filtered analog receive signal is then digitized by an analog to digital converter (ADC), phase aligned across the receive array by digital receive beamformer 126 to steer and focus the receive beam to the CW gate position. The phase aligned receive signals may then be summed across all receive elements to complete the receive beamformation.

The RF output of the digital receive beamformer 126 may be referred to as a receive beam. The receive beam may then be demodulated by digital complex demodulator 104 to shift the CW center frequency down to baseband. The IQ (in-phase and quadrature phase) output samples of complex demodulator 104 are time integrated by digital accumulator 106 to increase SNR. Time integration may also be performed by, e.g., a very long low-pass or band-pass filter. Preferably, the cut-off frequency of the low-pass filter or the upper cut-off frequency of the band-pass filter is set to the maximum Doppler shift frequency expected given the carrier frequency and maximum velocity of interest.

The output of accumulator 150 can be sampled at regular intervals (decimated) and sent to off-chip processor 180 for FFT and further processing to extract velocity information.

In some embodiments, the time gain compensation stage of the analog front end may be turned off for CW mode, because echo to a CW beam is not range or depth specific. To suppress low frequency noise and any DC drift, an analog high-pass filter may be set to the highest cut-off frequency option that is below the center frequency. To suppress thermal noise, an analog anti-aliasing filter cut off may be set to the lowest frequency option that is above the center frequency. The ADC sampling rate may be set to the highest frequency option possible to minimize thermal noise aliased into the CW band.

In all embodiments, receive beamformation for CW Mode is static, i.e., time and/or depth independent. This static receive beamformation can contrast with dynamic beamformation, such as the kind used for the pulsed wave (PW) modes. An on-chip delay computer 124 may create transmit and receive delay (or phase) values for each transmit and receive element.

In some embodiments, the digital receive beamformer 123 can form multiple receive beams in parallel using a digitized received echo. The use of digital circuits can advantageously allow for parallel processing paths. This is beneficial because parallel paths using an analog system can be prohibitively expensive, due to, among other factors, a need for an analog memory that can be read multiple times from the same port. The steering angle and/or focal depth of the parallel receive beams can be distinct, each corresponding to a distinct gate position. The downstream complex demodulator, accumulator and decimator 150, and the off-chip FFT blocks may process individual receive beams independently, which may enable iSoC 100 to provide flow information from multiple gate positions.

For embodiments involving multiple parallel receive beams, the transmit beam may be widened to support the angular span of the parallel receive beams. This can be done either by reducing the transmit aperture relative to the receive aperture size, or by setting the transmit focus deeper than the user's CW gate depth or setting it behind the aperture, resulting in a negative focal depth, to mimic a virtual point source.

For a single receive beam embodiment, the receive beam angle and focal depth may be set to match a user specified CW gate angle and depth. For embodiments using multiple parallel receive beams, the beam angle and focal depths of parallel beams can be distributed over a grid at and around a user-specified CW gate. In this case, a CW spectra of parallel beams allow automatic detection of the position of the maximum velocity flow, improving the positional and spectral accuracy, and shortening workflow and exam time. In some embodiments, the receive center frequency (i.e., the complex demodulation frequency) may be set to match the transmit center frequency.

Two independent accumulators working in parallel can be configured as a complex accumulator of the I and Q samples of the output of complex demodulator 150. The complex accumulator outputs may then be sampled and stored, and the accumulator may be reset, e.g., at regular time intervals. The accumulator output samples are the inputs of an off-ASIC FFT processor.

Alternatively, two identical long low-pass filters working in parallel can be configured as a complex accumulator. These low-pass filters may be baseband filters (“BBF”). The BBF may have a uniform window, e.g., a box-car filter, where all coefficients are one. The BBF may have a tapered window, such as a Hamming window. The complex output of the BBF is sampled and stored. The sampling may be down-sampling, and the down sampling rate may be equal to the length of the BBF window, for a non-overlapping window, or the down sampling rate may be half the length of the window, for half overlapping windows.

In accordance with some embodiments, the output of accumulator 150 may be sampled at every 244.14μsec (½¹²=¼,096 sec). At this output rate (4,096 Hz) a 128-point FFT can be generated 32 times a second over 128-sample long non-overlapping windows, or 64 times a second over 128-sample long half-overlapping windows. The frequency resolution of FFTs, i.e., the number of FFT points, can be traded off for the temporal resolution of FFTs, i.e., the number of FFTs per second over independent windows or windows that are at most half overlapping, and vice versa. Persons having skill in the art will understand that other values may also be used.

The accumulator length in number of input samples is determined by the accumulation duration (244.14μsec), and the ADC sampling rate Fs. If the transmit and receive center frequency is 2 MHz, Fs of 16 MHz would be adequate for beamformer delay quantization. The accumulator length would then be (½\hat{ }12) sec/FFT sample *16e6 input samples/sec =3,906 input samples/FFT sample. Such a long accumulator improves SNR by ~36 dB (10*log10(3906)=35.9). Optionally the ADC sampling rate can be set higher, e.g., to 64 MHz for an additional 6 dB SNR improvement (3 dB for each doubling of the ADC rate). To reflect the increase in SNR, the bit-width of the accumulator output is increased. For example, for this example the bit widths of I and Q samples are increased each by 7 bits (log(64e 6/2\hat{ }12)/log (2)/2 =7).

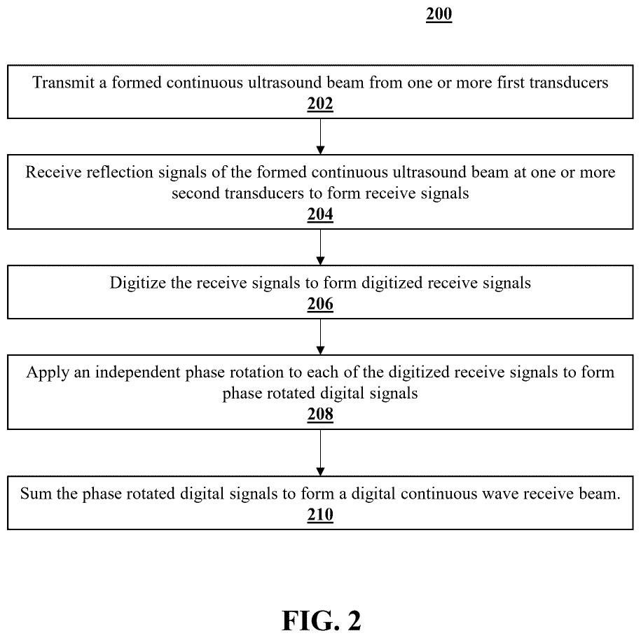

Turning now to FIG. 2, a flow diagram of a method 200 for continuous wave ultrasound imaging in accordance with some embodiments, is shown. The method 200 is performed at a computing circuit of an ultrasound imaging device, such as iSoC 100 of FIG. 1. As the computing circuit, a continuous ultrasound beam is transmitted 202 from one or more first transducers 171, such as matrix array transducers 170 of FIG. 1. Reflection signals of the formed continuous ultrasound beam are then received 204 at one or more second transducers 172 to form receive signals. The receive signals are then digitized to form digitized receive signals. An independent phase rotation is then applied 206 to each of the digitized receive signals to form phase rotated digital signals. The phase rotated digital signals are then summed 208 to form a digital continuous wave receive beam.

In some embodiments, the one or more first transducers are programmed to perform only a transmit function. In some embodiments, the one or more second transducers are programmed to perform only a receive function. In some embodiments, the one or more first transducers are different from the one or more second transducers. In some embodiments, the reflection signals are amplified before they are digitized. In some embodiments, the continuous wave receive beam are demodulated down to baseband signals. In some embodiments, the baseband signals are low pass filtered and decimated to form input samples of a spectral analyzer.

In some embodiments, the one or more first transducers and the one or more second transducers form a matrix array of transducers. In some embodiments, the one or more first transducers and the one or more second transducers are included in a matrix array of transducers. In some embodiments, the one or more first transducers is a first half of the matrix array of transducers. In some embodiments, the one or more second transducers is a second half of the matrix array of transducers.

In some embodiments, independent phase rotation can be applied to continuous wave excitation of each of the one or more first transducers, before transmitting the continuous ultrasound beam, to steer the continuous ultrasound beam substantially along a line of sight of interest and to focus at a depth of interest.

In some embodiments, the applying the independent phase rotation to each of the digitized receive signals can be performed to steer the digitized receive signals substantially along a transmit line of sight and focus the digitized receive signals substantially at a transmit focus depth.

In some embodiments, the applying the independent phase rotation to each of the digitized receive signals can be performed multiple times in parallel to steer the digitized receive signals along multiple distinct angles and/or depths forming multiple continuous wave receive beams in parallel. In some embodiments, where at least one of the multiple continuous wave receive beams can be steered out of a standard azimuthal plane at a nonzero angle relative to an axis normal to a probe surface.

In some embodiments, the computing circuit can be an application specific integrated circuit (ASIC) such as iSoC 100. In some embodiments, the application specific integrated circuit (ASIC) can be integrated with the first one or more transducers 171 and the second one or more transducers 172.

The foregoing description, for purpose of explanation, has been described with reference to specific implementations. However, the illustrative discussions above are not intended to be exhaustive or to limit the scope of the claims to the precise forms disclosed. Many modifications and variations are possible in view of the above teachings. The implementations are chosen in order to best explain the principles underlying the claims and their practical applications, to thereby enable others skilled in the art to best use the implementations with various modifications as are suited to the particular uses contemplated.

### Illustration of Subject Technology as Clauses

Various examples of aspects of the disclosure are described as numbered clauses (1, 2, 3, etc.) for convenience. These are provided as examples, and do not limit the subject technology. Identifications of the figures and reference numbers are provided below merely as examples and for illustrative purposes, and the clauses are not limited by those identifications.

Clause 1. A method of forming a digital continuous wave ultrasound beam, comprising: at a computing circuit of an ultrasound imaging device: transmitting a formed continuous ultrasound beam from one or more first transducers; receiving reflection signals of the formed continuous ultrasound beam at one or more second transducers to form receive signals; digitizing the receive signals to form digitized receive signals; applying an independent phase rotation to each of the digitized receive signals to form phase rotated digital signals; and summing the phase rotated digital signals to form a digital continuous wave receive beam.

Clause 2. The method of Clause 1, wherein the one or more first transducers are programmed to perform only a transmit function.

Clause 3. The method of any of the preceding Clauses, wherein the one or more second transducers are programmed to perform only a receive function.

Clause 4. The method of any of the preceding Clauses, wherein the one or more first transducers are different from the one or more second transducers.

Clause 5. The method of any of the preceding Clauses, further comprising before digitizing, amplifying the reflection signals.

Clause 6. The method of any of the preceding Clauses, further comprising demodulating the continuous wave receive beam down to baseband signals.

Clause 7. The method of Clause 6, further comprising low pass filtering and decimating the baseband signals to form input samples of a spectral analyzer.

Clause 8. The method of any of the preceding Clauses, wherein the one or more first transducers and the one or more second transducers form a matrix array of transducers.

Clause 9. The method of any of the preceding Clauses, wherein the one or more first transducers and the one or more second transducers are included in a matrix array of transducers.

Clause 10. The method of Clause 8, wherein the one or more first transducers is a first half of the matrix array of transducers.

Clause 11. The method of Clause 8, wherein the one or more second transducers is a second half of the matrix array of transducers.

Clause 12. The method of any of the preceding Clauses, further comprising, before transmitting the formed continuous ultrasound beam, applying independent phase rotation to continuous wave excitation of each of the one or more first transducers to steer the formed continuous ultrasound beam substantially along a line of sight of interest and focused at a depth of interest.

Clause 13. The method of any of the preceding Clauses, wherein the applying the independent phase rotation to each of the digitized receive signals is performed to steer the digitized receive signals substantially along a transmit line of sight and focus the digitized receive signals substantially at a transmit focus depth.

Clause 14. The method of any of the preceding Clauses, wherein the applying the independent phase rotation to each of the digitized receive signals is performed multiple times in parallel to steer the digitized receive signals along multiple distinct angles and/or depths forming multiple continuous wave receive beams in parallel.

Clause 15. The method of Clause 14, where at least one of the multiple continuous wave receive beams is steered out of a standard azimuthal plane at a nonzero angle relative to an axis normal to a probe surface.

Clause 16. The method of any of the preceding Clauses, wherein the computing circuit is an application specific integrated circuit (ASIC).

Clause 17. The method of Clause 16, wherein the application specific integrated circuit (ASIC) is integrated with the first one or more transducers and the second one or more transducers.

Clause 18. An ultrasound imaging device, comprising: a computing circuit; one or more processors; memory; and one or more programs stored in the memory and configured for execution by the one or more processors, the one or more programs comprising instructions for: transmitting a formed continuous ultrasound beam from one or more first transducers; receiving reflection signals of the formed continuous ultrasound beam at one or more second transducers to form receive signals; digitizing the receive signals to form digitized receive signals; applying an independent phase rotation to each of the digitized receive signals to form phase rotated digital signals; and summing the phase rotated digital signals to form a digital continuous wave receive beam.

Clause 19. The device of Clause 18, wherein the one or more first transducers are programmed to perform only a transmit function.

Clause 20. The device of any of Clauses 18 to 19, wherein the one or more second transducers are programmed to perform only a receive function.

Clause 21. The device of any of Clauses 18 to 20, wherein the one or more first transducers are different from the one or more second transducers.

Clause 22. The device of any of Clauses 18 to 21, wherein the operations further comprise before digitizing, amplifying the reflection signals.

Clause 23. The device of any of Clauses 18 to 22, wherein the operations further comprise demodulating the continuous wave receive beam down to baseband signals.

Clause 24. The device of Clause 23, wherein the operations further comprise low pass filtering and decimating the baseband signals to form input samples of a spectral analyzer.

Clause 25. The device of any of Clauses 18 to 24, wherein the one or more first transducers and the one or more second transducers form a matrix array of transducers.

Clause 26. The device of any of Clauses 18 to 25, wherein the one or more first transducers and the one or more second transducers are included in a matrix array of transducers.

Clause 27. The device of Clause 25, wherein the one or more first transducers is a first half of the matrix array of transducers.

Clause 28. The device of Clause 25, wherein the one or more second transducers is a second half of the matrix array of transducers.

Clause 29. The device of any of Clauses 18 to 28, further comprising, before transmitting the formed continuous ultrasound beam, applying independent phase rotation to continuous wave excitation of each of the one or more first transducers to steer the formed continuous ultrasound beam substantially along a line of sight of interest and focused at a depth of interest.

Clause 30. The device of any of Clauses 18 to 29, wherein the applying the independent phase rotation to each of the digitized receive signals is performed to steer the digitized receive signals substantially along a transmit line of sight and focus the digitized receive signals substantially at a transmit focus depth.

Clause 31. The device of any of Clauses 18 to 30, wherein the applying the independent phase rotation to each of the digitized receive signals is performed multiple times in parallel to steer the digitized receive signals along multiple distinct angles and/or depths forming multiple continuous wave receive beams in parallel.

Clause 32. The device of Clause 31, where at least one of the multiple continuous wave receive beams is steered out of a standard azimuthal plane at a nonzero angle relative to an axis normal to a probe surface.

Clause 33. The device of any of Clauses 18 to 32, wherein the computing circuit is an application specific integrated circuit (ASIC).

Clause 34. The device of Clause 33, wherein the application specific integrated circuit (ASIC) is integrated with the first one or more transducers and the second one or more transducers.

### Further Considerations

In some embodiments, any of the clauses herein may depend from any one of the independent clauses or any one of the dependent clauses. In one aspect, any of the clauses (e.g., dependent or independent clauses) may be combined with any other one or more clauses (e.g., dependent or independent clauses). In one aspect, a claim may include some or all of the words (e.g., steps, operations, means or components) recited in a clause, a sentence, a phrase or a paragraph. In one aspect, a claim may include some or all of the words recited in one or more clauses, sentences, phrases or paragraphs. In one aspect, some of the words in each of the clauses, sentences, phrases or paragraphs may be removed. In one aspect, additional words or elements may be added to a clause, a sentence, a phrase or a paragraph. In one aspect, the subject technology may be implemented without utilizing some of the components, elements, functions or operations described herein. In one aspect, the subject technology may be implemented utilizing additional components, elements, functions or operations.

As used herein, the word “module” refers to logic embodied in hardware or firmware, or to a collection of software instructions, possibly having entry and exit points, written in a programming language, such as, for example C++. A software module may be compiled and linked into an executable program, installed in a dynamic link library, or may be written in an interpretive language such as BASIC. It will be appreciated that software modules may be callable from other modules or from themselves, and/or may be invoked in response to detected events or interrupts. Software instructions may be embedded in firmware, such as an EPROM or EEPROM. It will be further appreciated that hardware modules may be comprised of connected logic units, such as gates and flip-flops, and/or may be comprised of programmable units, such as programmable gate arrays or processors. The modules described herein are preferably implemented as software modules but may be represented in hardware or firmware.

It is contemplated that the modules may be integrated into a fewer number of modules. One module may also be separated into multiple modules. The described modules may be implemented as hardware, software, firmware or any combination thereof. Additionally, the described modules may reside at different locations connected through a wired or wireless network, or the Internet.

In general, it will be appreciated that the processors can include, by way of example, computers, program logic, or other substrate configurations representing data and instructions, which operate as described herein. In other embodiments, the processors can include controller circuitry, processor circuitry, processors, general purpose single-chip or multi-chip microprocessors, digital signal processors, embedded microprocessors, microcontrollers and the like.

Furthermore, it will be appreciated that in one embodiment, the program logic may advantageously be implemented as one or more components. The components may advantageously be configured to execute on one or more processors. The components include, but are not limited to, software or hardware components, modules such as software modules, object-oriented software components, class components and task components, processes methods, functions, attributes, procedures, subroutines, segments of program code, drivers, firmware, microcode, circuitry, data, databases, data structures, tables, arrays, and variables.

The foregoing description is provided to enable a person skilled in the art to practice the various configurations described herein. While the subject technology has been particularly described with reference to the various figures and configurations, it should be understood that these are for illustration purposes only and should not be taken as limiting the scope of the subject technology.

There may be many other ways to implement the subject technology. Various functions and elements described herein may be partitioned differently from those shown without departing from the scope of the subject technology. Various modifications to these configurations will be readily apparent to those skilled in the art, and generic principles defined herein may be applied to other configurations. Thus, many changes and modifications may be made to the subject technology, by one having ordinary skill in the art, without departing from the scope of the subject technology.

It is understood that the specific order or hierarchy of steps in the processes disclosed is an illustration of exemplary approaches. Based upon design preferences, it is understood that the specific order or hierarchy of steps in the processes may be rearranged. Some of the steps may be performed simultaneously. The accompanying method claims present elements of the various steps in a sample order, and are not meant to be limited to the specific order or hierarchy presented.

As used herein, the phrase “at least one of” preceding a series of items, with the term “and” or “or” to separate any of the items, modifies the list as a whole, rather than each member of the list (i.e., each item). The phrase “at least one of” does not require selection of at least one of each item listed; rather, the phrase allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

Terms such as “top,” “bottom,” “front,” “rear” and the like as used in this disclosure should be understood as referring to an arbitrary frame of reference, rather than to the ordinary gravitational frame of reference. Thus, a top surface, a bottom surface, a front surface, and a rear surface may extend upwardly, downwardly, diagonally, or horizontally in a gravitational frame of reference.

Furthermore, to the extent that the term “include,” “have,” or the like is used in the description or the claims, such term is intended to be inclusive in a manner similar to the term “comprise” as “comprise” is interpreted when employed as a transitional word in a claim.

As used herein, the term “about” is relative to the actual value stated, as will be appreciated by those of skill in the art, and allows for approximations, inaccuracies and limits of measurement under the relevant circumstances. In one or more aspects, the terms “about,” “substantially,” and “approximately” may provide an industry-accepted tolerance for their corresponding terms and/or relativity between items, such as a tolerance of from less than one percent to 10 percent of the actual value stated, and other suitable tolerances.

As used herein, the term “comprising” indicates the presence of the specified integer(s), but allows for the possibility of other integers, unspecified. This term does not imply any particular proportion of the specified integers. Variations of the word “comprising,” such as “comprise” and “comprises,” have correspondingly similar meanings.

The word “exemplary” is used herein to mean “serving as an example, instance, or illustration.” Any embodiment described herein as “exemplary” is not necessarily to be construed as preferred or advantageous over other embodiments.

A reference to an element in the singular is not intended to mean “one and only one” unless specifically stated, but rather “one or more.” Pronouns in the masculine (e.g., his) include the feminine and neuter gender (e.g., her and its) and vice versa. The term “some” refers to one or more. Underlined and/or italicized headings and subheadings are used for convenience only, do not limit the subject technology, and are not referred to in connection with the interpretation of the description of the subject technology. All structural and functional equivalents to the elements of the various configurations described throughout this disclosure that are known or later come to be known to those of ordinary skill in the art are expressly incorporated herein by reference and intended to be encompassed by the subject technology. Moreover, nothing disclosed herein is intended to be dedicated to the public regardless of whether such disclosure is explicitly recited in the above description.

Although the detailed description contains many specifics, these should not be construed as limiting the scope of the subject technology but merely as illustrating different examples and aspects of the subject technology. It should be appreciated that the scope of the subject technology includes other embodiments not discussed in detail above. Various other modifications, changes and variations may be made in the arrangement, operation and details of the method and apparatus of the subject technology disclosed herein without departing from the scope of the present disclosure. In addition, it is not necessary for a device or method to address every problem that is solvable (or possess every advantage that is achievable) by different embodiments of the disclosure in order to be encompassed within the scope of the disclosure. The use herein of “can” and derivatives thereof shall be understood in the sense of “possibly” or “optionally” as opposed to an affirmative capability.

1. A method of forming a digital continuous wave ultrasound beam, comprising:
at a computing circuit of an ultrasound imaging device:
transmitting a formed continuous ultrasound beam from one or more first transducers;
receiving reflection signals of the formed continuous ultrasound beam at one or more second transducers to form receive signals;
digitizing the receive signals to form digitized receive signals;
applying an independent phase rotation to each of the digitized receive signals to form phase rotated digital signals; and
summing the phase rotated digital signals to form a digital continuous wave receive beam.

2. The method of claim 1, wherein the one or more first transducers are programmed to perform only a transmit function.

3. The method of claim 1, wherein the one or more second transducers are programmed to perform only a receive function.

4. The method of claim 1, wherein the one or more first transducers are different from the one or more second transducers.

5. The method of claim 1, further comprising before digitizing, amplifying the reflection signals.

6. The method of claim 1, further comprising demodulating the continuous wave receive beam down to baseband signals.

7. The method of claim 1, further comprising, before transmitting the formed continuous ultrasound beam, applying independent phase rotation to continuous wave excitation of each of the one or more first transducers to steer the formed continuous ultrasound beam substantially along a line of sight of interest and focused at a depth of interest.

8. The method of claim 1, wherein the applying the independent phase rotation to each of the digitized receive signals is performed to steer the digitized receive signals substantially along a transmit line of sight and focus the digitized receive signals substantially at a transmit focus depth.

9. The method of claim 1, wherein the applying the independent phase rotation to each of the digitized receive signals is performed multiple times in parallel to steer the digitized receive signals along multiple distinct angles and/or depths forming multiple continuous wave receive beams in parallel.

10. The method of claim 1, wherein the computing circuit is an application specific integrated circuit (ASIC).

11. An ultrasound imaging device, comprising:
a computing circuit;
one or more processors;
memory; and
one or more programs stored in the memory and configured for execution by the one or more processors, the one or more programs comprising instructions for:
transmitting a formed continuous ultrasound beam from one or more first transducers;
receiving reflection signals of the formed continuous ultrasound beam at one or more second transducers to form receive signals;
digitizing the receive signals to form digitized receive signals;
applying an independent phase rotation to each of the digitized receive signals to form phase rotated digital signals; and
summing the phase rotated digital signals to form a digital continuous wave receive beam.

12. The device of claim 11, wherein the one or more first transducers are programmed to perform only a transmit function.

13. The device of claim 11, wherein the one or more second transducers are programmed to perform only a receive function.

14. The device of claim 11, wherein the one or more first transducers are different from the one or more second transducers.

15. The device of claim 11, wherein the operations further comprise before digitizing, amplifying the reflection signals.

16. The device of claim 11, wherein the operations further comprise demodulating the continuous wave receive beam down to baseband signals.

17. The device of claim 11, further comprising, before transmitting the formed continuous ultrasound beam, applying independent phase rotation to continuous wave excitation of each of the one or more first transducers to steer the formed continuous ultrasound beam substantially along a line of sight of interest and focused at a depth of interest.

18. The device of claim 11, wherein the applying the independent phase rotation to each of the digitized receive signals is performed to steer the digitized receive signals substantially along a transmit line of sight and focus the digitized receive signals substantially at a transmit focus depth.

19. The device of claim 11, wherein the applying the independent phase rotation to each of the digitized receive signals is performed multiple times in parallel to steer the digitized receive signals along multiple distinct angles and/or depths forming multiple continuous wave receive beams in parallel.

20. The device of claim 11, wherein the computing circuit is an application specific integrated circuit (ASIC).
